(19) United States
(12) Patent Application Publication
(10) Pub. No.: US 2019/0336240 A1
(43) Pub. Date: Nov. 7, 2019

(54) METHOD FOR ABLATION TREATMENT OF ILEAL MUCOSA

(71) Applicant: OLYMPUS CORPORATION, Tokyo (JP)

(72) Inventor: Ryosuke ITO, Tokyo (JP)

(73) Assignee: OLYMPUS CORPORATION, Tokyo (JP)

(21) Appl. No.: 15/972,622

(22) Filed: May 7, 2018

(51) Int. Cl. A61B 90/00; A61B 18/04; A61B 18/08; A61B 18/14; A61B 18/24

(52) U.S. Cl. CPC A61B 90/39; A61B 18/042; A61B 2017/320069; A61B 18/1492; A61B 18/24; A61B 18/082

(57) ABSTRACT

An ileal-mucosa ablation treatment method including: setting a length of a treatment area in an ileal mucosa, wherein the length of the treatment area is set on the basis of at least one of patient characteristics and pathophysiological conditions; marking an end point of the treatment area in the ileal mucosa; and performing ablation treatment on the ileal mucosa, wherein, in the performing ablation treatment, ablation treatment is performed on the ileal mucosa from a start point to the end point while changing treatment positions toward the end point from the start point, and the start point is at a position that is separated from the end point in a longitudinal direction of the ileum by the set length.

### TECHNICAL FIELD

The present invention relates to an ileal-mucosa ablation treatment method.

### BACKGROUND ART

In the related art, there is a known method in which ablation treatment is performed on the mucosa of the duodenum or the jejunum as a method for treating obesity and type-2 diabetes (see, for example, Patent Literature 1). By performing ablation treatment on the mucosa of the duodenum or the jejunum, it is possible to improve obesity and type-2 diabetes by inhibiting absorption of nutrients and fat.

Ablation treatment of the ileal mucosa is also expected to have an improvement effect on obesity and type-2 diabetes. Each segments of the gastrointestinal tract have different functions, and therapeutic mechanisms also differ in accordance with the segments on which ablation treatment is performed. In the case of the ileum, in which bile acid is absorbed, it is presumed that a therapeutic effect is achieved as a result of suppressing bile-acid absorption by means of ablation treatment performed on the ileal mucosa. In order to control the suppression of bile-acid absorption in the ileum, it is important to control the treatment area in the ileal mucosa. Patent Literature 1 does not disclose controlling the treatment area or controlling the therapeutic effect by doing so.

### CITATION LIST

### Patent Literature

{PTL 1}

U.S. Pat. No. 8,641,711

### SUMMARY OF INVENTION

An aspect of the present invention is a method for performing ablation treatment on an ileal mucosa that covers an inner surface of the ileum, the ileal-mucosa ablation treatment method comprising: setting a length of a treatment area in the ileal mucosa, wherein the length of the treatment area is set on the basis of at least one of patient characteristics and pathophysiological conditions; marking an end point of the treatment area in the ileal mucosa; and performing ablation treatment on the ileal mucosa, wherein, in the performing ablation treatment, ablation treatment is performed on the ileal mucosa from a start point to the end point while changing the treatment position toward the end point from the start point, and the start point is at a position that is separated from the end point in a longitudinal direction of the ileum by the set length.

### BRIEF DESCRIPTION OF DRAWINGS

FIG. 1 is an overall configuration diagram of an ablation system used in an ablation treatment method according to an embodiment of the present invention.

FIG. 2 is a flowchart showing the ileal-mucosa ablation treatment method according to the embodiment of the present invention.

FIG. 3 is a diagram for explaining an end point and a start point determined in the ileum.

FIG. 4A is a diagram showing an example of an ablation pattern and ablation density applied to the ileal mucosa by an ablation treatment tool.

FIG. 4B is a diagram showing another example of an ablation pattern and ablation density applied to the ileal mucosa by the ablation treatment tool.

FIG. 4C is a diagram showing another example of an ablation pattern and ablation density applied to the ileal mucosa by the ablation treatment tool.

FIG. 4D is a diagram showing another example of an ablation pattern and ablation density applied to the ileal mucosa by the ablation treatment tool.

FIG. 4E is a diagram showing another example of an ablation pattern and ablation density applied to the ileal mucosa by the ablation treatment tool.

FIG. 5A is a configuration diagram of a marking apparatus used to mark the end point and the start point.

FIG. 5B is a configuration diagram of a modification of the marking apparatus in FIG. 5A.

FIG. 6A is a diagram for explaining an end-point marking step.

FIG. 6B is a diagram for explaining a start-point determining step.

FIG. 6C is a diagram for explaining a start-point marking step.

FIG. 6D is a diagram for explaining a ablation treatment step.

FIG. 6E is a diagram for explaining the ablation treatment step.

FIG. 7 is a configuration diagram of another modification of the marking apparatus.

FIG. 8 is a diagram for explaining a modification of the start-point determining step.

FIG. 9 is a diagram showing a modification of marking of the end point and start point.

FIG. 10 is a diagram showing modifications of the start point and end point determined in the ileum.

### DESCRIPTION OF EMBODIMENT

An ablation treatment method according to an embodiment of the present invention will be described with reference to the drawings.

The ablation treatment method according to this embodiment is applied to treat a patient having metabolic diseases (metabolic syndrome) such as type 2 diabetes or the like. The small intestine is constituted of the jejunum connected to the duodenum and the ileum connected to the large intestine. The ileum has a function of absorbing bile acid. Bile acid is a component of bile and has the function of controlling glucose metabolism. As a result of ablation treatment of the ileal mucosa, which covers the inner surface of the ileum, the bile-acid absorption function of the ileum is adjusted, and thus, it is possible to improve metabolic disease.

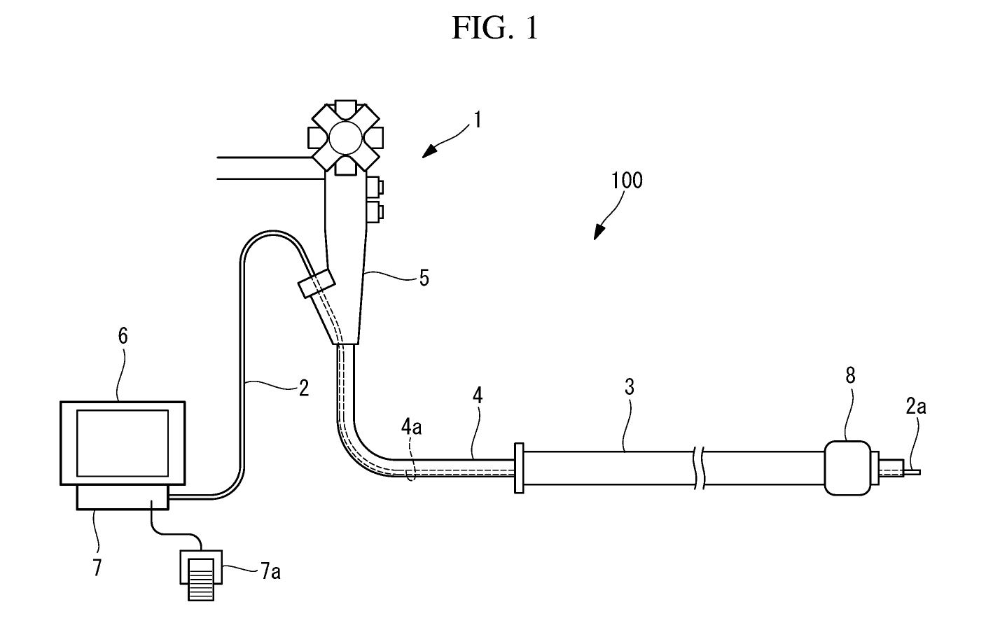

FIG. 1 shows an ablation system 100 used in the ablation treatment method according to this embodiment. As shown in FIG. 1, the ablation system 100 is provided with an endoscope 1, an ablation treatment tool 2, and an overtube 3.

The endoscope 1 is provided with an elongated and flexible inserted portion 4 and a handle portion 5 that is connected to a base end of the inserted portion 4 and that is used to manipulate a bending portion, etc. of the inserted portion 4. The inserted portion 4 has a treatment tool channel 4a that passes therethrough in a longitudinal direction and into which the ablation treatment tool 2 is inserted. The endoscope 1 is connected to a monitor 6, and an endoscope image acquired by the endoscope 1 is displayed on the monitor 6.

The ablation treatment tool 2 is an elongated and flexible device and discharges energy from a distal-end portion 2a. Ablation treatment of a mucosa is performed by means of the energy supplied to the mucosa from the distal-end portion 2a. The energy refers to, for example, argon plasma energy, laser energy, radio-frequency electrical energy, high-frequency electrical energy, heat energy, or ultrasonic wave energy. “Ablation treatment” refers to treatment in which epithelial cells present in the ileal mucosa are denatured or in which the epithelial cells are removed from the ileal mucosa. “Denature” refers to a cell alteration that is severe enough to cause a loss of the bile-acid absorption function originally performed by the epithelial cells.

Regarding the ablation treatment tool 2, there are a non-contact type, with which ablation treatment is performed on the mucosa in a state in which the distal-end portion 2a is separated from the mucosa, and a contact type, with which ablation treatment is performed on the mucosa in a state in which the distal-end portion 2a is in contact with the mucosa.

In the case of the non-contact type, argon plasma energy or laser energy is emitted from the distal-end portion 2a.

In the case of the contact type, radio-frequency electrical energy, high-frequency electrical energy, heat energy or ultrasonic energy are discharged from the distal-end portion 2a. For example, said energy is discharged from an electrode or an ultrasonic transducer provided at a surface of a balloon or a plate, or is discharged from a rake-type or wire-type electrode.

The ablation treatment tool 2 is connected to a controller 7 and is controlled by means of the controller 7. A foot pedal 7a is connected to the controller 7. By manipulating the foot pedal 7a with his/her foot, an operator can control starting of the energy output from the ablation treatment tool 2 and stopping of the energy output therefrom.

The overtube 3 is an elongated flexible and circular tube-like member and has an inner diameter that is greater than the outer diameter of the inserted portion 4. The inserted portion 4 can be inserted into the overtube 3 along the longitudinal direction thereof, and it is possible to move the inserted portion 4 and the overtube 3 relative to each other in the longitudinal direction. At a distal-end portion of the overtube 3, a balloon 8 for fixing the overtube 3 to the gastrointestinal tract is provided. The balloon 8 wraps around the entire circumference of the overtube 3 over an outer circumferential surface thereof and can be inflated and deflated in radial directions of the overtube 3. Inflation and deflation of the balloon 8 are controlled by supplying a fluid into the balloon 8 via a flow channel (not shown) provided in the overtube 3 and discharging the fluid therefrom. The overtube 3 serves as an auxiliary tool for allowing the endoscope to reach the ileum. The small-intestine endoscope procedure employing the overtube 3 indicated in this embodiment is referred to as single-balloon small-intestine endoscopy. Alternatively, the ablation system may be a system employing double-balloon small-intestine endoscopy or spiral small-intestine endoscopy.

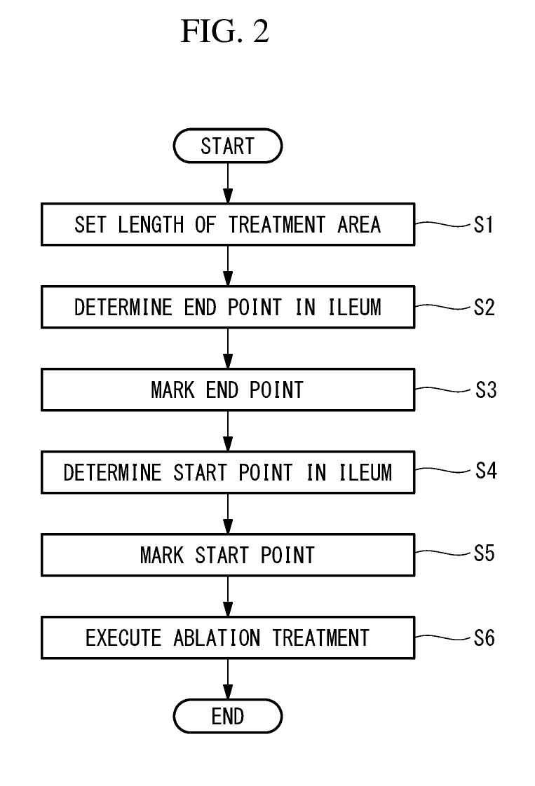

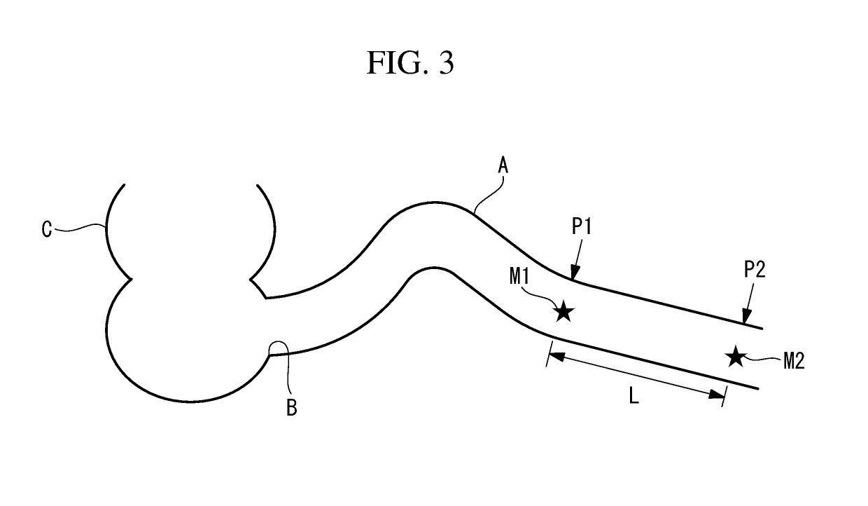

As shown in FIGS. 2 and 3, the ablation treatment method according to this embodiment includes: a length setting step S1 of setting a length L of a treatment area in the ileal mucosa; an end-point determining step S2 of determining an end point P1 of the treatment area; an end-point marking step S3 of marking the end point P1; a start-point determining step S4 of determining a start point P2 of the treatment area; a start-point marking step S5 of marking the start point P2; and a ablation treatment step S6 of performing ablation treatment on the treatment area from the start point P2 to the end point P1 in the ileal mucosa.

In the length setting step S1, the operator sets the length L of the treatment area on the basis of at least one of patient characteristics and pathophysiological conditions.

The patient characteristics refer to, for example, age, height, weight, BMI (body-mass index), visceral fat mass, blood components (HbA1c, HDL, LDL, insulin, etc.), diabetes history, treatment/medication history, insulin-medication use history, and the intestinal length measured in a preliminary examination employing CT or MRI.

The patient pathophysiological conditions refer to, for example, intestinal-bacteria composition analysis results, analysis results of the bile-acid concentration and compositions in blood or stool, bile-acid malabsorption evaluation results of a SeHCAT test, the presence/absence of new bile-acid-synthesis promotion or carbohydrate metabolism improvement results when a bile-acid absorption suppression drug is administered, and the presence/absence of new bile-acid-synthesis promotion or carbohydrate metabolism improvement results when bile-acid suppression equipment is temporarily disposed in the ileum A.

The total length of the ileum A is generally approximately 3 m. In the ileum A, a bile-acid absorption region is located within an area of approximately 100 cm from the ileocecal valve B. The ileocecal valve B is a valve that is present in a terminal portion of the ileum A, which is a connecting portion between the ileum A and the large intestine C, and is also referred to as the Bauhin valve. Therefore, the length L is set to be equal to or less than 100 cm.

In order to achieve an improvement effect in metabolic diseases by means of ablation treatment on the ileal mucosa, it is considered that an appropriate level of suppression is approximately 10% of the total amount of bile acid absorbed in the ileum A. The length L is a length that corresponds to a desired size of the treatment area in the ileal mucosa at which an improvement effect of metabolic diseases is achieved. The preferred range of the length L is 10 to 40 cm.

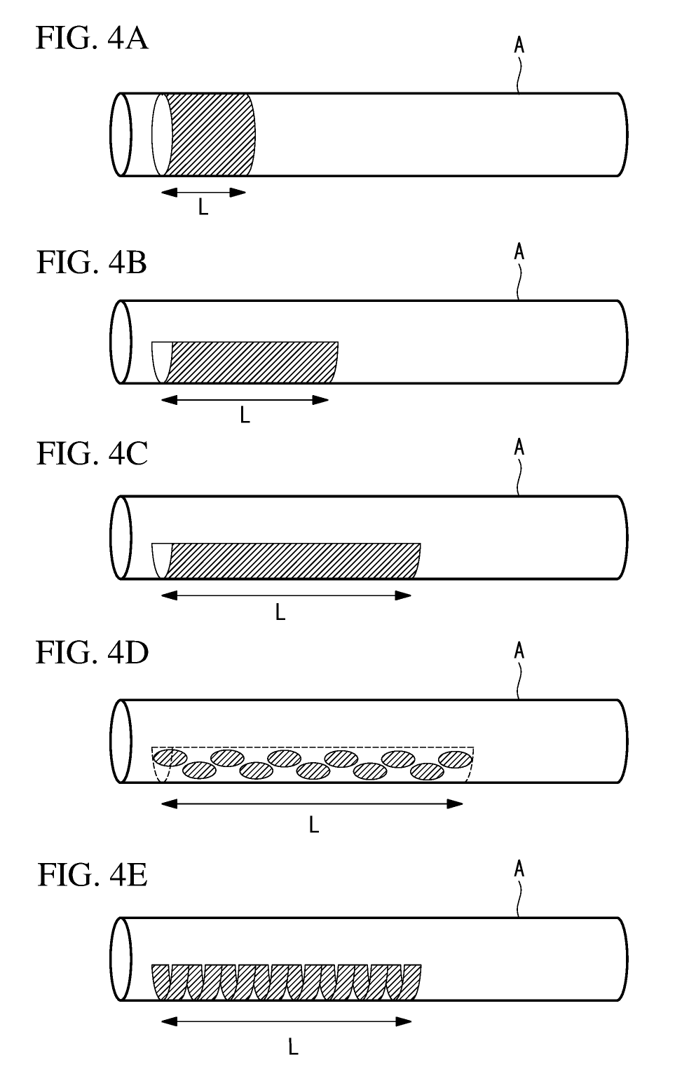

The length L is determined in accordance with the density of ablation applied to the ileal mucosa by the ablation treatment tool 2. FIGS. 4A to 4E show examples of the ablation density. The ablation density is determined in accordance with the structure of a distal-end portion 2a of the ablation treatment tool 2 and spacings between treatment positions.

As shown in FIG. 4A, in the case in which ablation treatment is performed on the ileal mucosa over the entire circumference thereof, the length L is set so that the product of the circumferential length of the ileum and the length L is equal to the desired size of the treatment area.

As shown in FIG. 4B, in the case in which ablation treatment is performed on the ileal mucosa over half the circumference thereof, the length L is set so that the product of half the circumferential length of the ileum and the length L is equal to the desired size of the treatment area.

As shown in FIG. 4C, in the case in which ablation treatment is performed on the ileal mucosa over ⅓ of the circumference thereof, the length L is set so that the product of ⅓ of the circumferential length of the ileum A and the length L is equal to the desired size of the treatment area.

As shown FIGS. 4D and 4E, in the case in which ablation treatment is performed on the ileal mucosa at positions with spacings therebetween, the length L is set so that the product of the ablation density per unit length in the longitudinal direction of the ileum A and the length L is equal to the desired size of the treatment area.

Next, in the end-point determining step S2, the end point P1 is determined in the bile-acid absorption region on the basis of the inserted length of the inserted portion 4 from the ileocecal valve B. The end point P1 is determined in consideration of the length L so that the start point P2, which is subsequently determined so as to be closer to the jejunum than the end point P1 is, is also positioned in the bile-acid absorption region.

Before or after the length setting step S1, the inserted portion 4 is inserted, together with the overtube 3, from the anus so as to reach the ileum A via the large intestine C. After determining the end point P1, the overtube 3 is positioned at a position at which the end point P1 is disposed in the viewing field of the inserted portion 4 protruding from the distal end of the overtube 3, and the overtube 3 is fixed to the ileum A by inflating the balloon 8. By doing so, the determined end point P1 is not displaced.

The inserted portion 4 may be inserted orally so as to reach the ileum A via the duodenum, instead of being inserted from the anus so as to reach the ileum A via the large intestine C.

The end point P1 may be determined on the basis of an endoscope image of the small-intestine interior acquired by the endoscope 1. The interval of circular folds in the ileum A is less than the interval of circular folds in the jejunum. Therefore, it is possible to determine the end point P1 in the bile-acid absorption region in the ileum A by judging whether the ileum A or the jejunum is in the image on the basis of the interval of circular folds in the endoscope image.

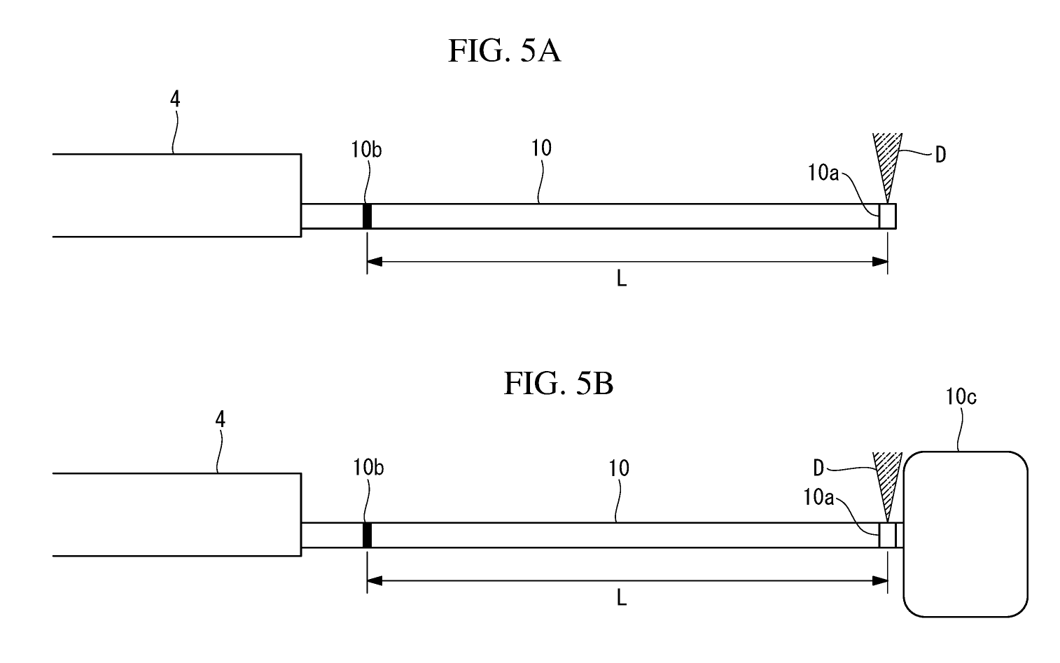

Next, in the end-point marking step S3, the ileal mucosa at the end point P1 is marked with a dye D by using a marking apparatus 10. As shown in FIG. 5A, the marking apparatus 10 is an elongated device to be inserted into the treatment tool channel 4a of the inserted portion 4. The marking apparatus 10 includes a marking portion 10a and an positioning mark 10b. The marking portion 10a is provided at a distal-end portion of the marking apparatus 10 and sprays the dye D. The positioning mark 10b is provided at a position that is separated from the marking portion 10a toward the base end by the length L.

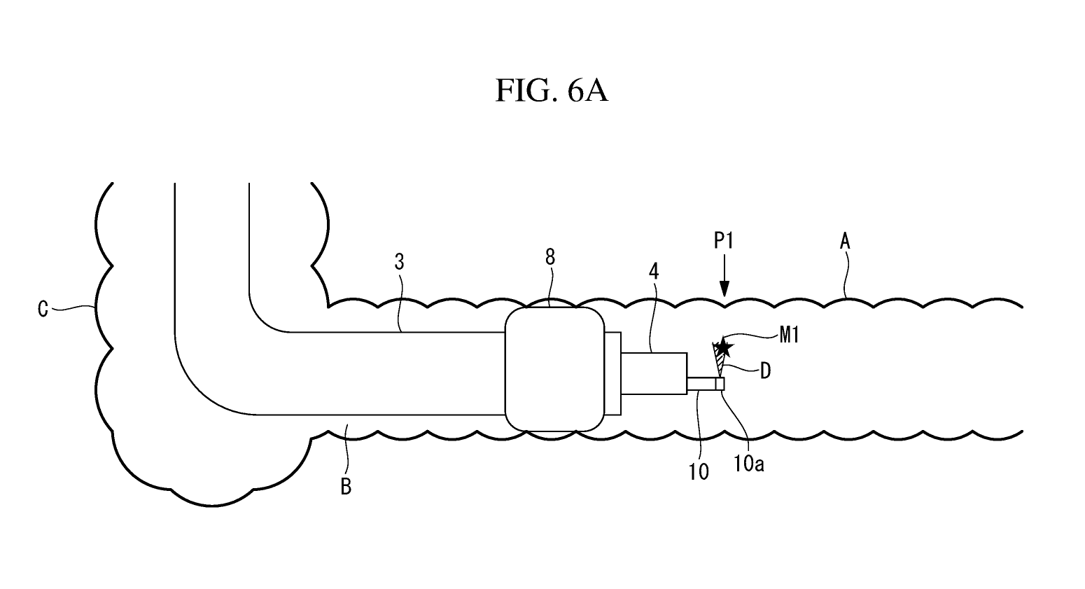

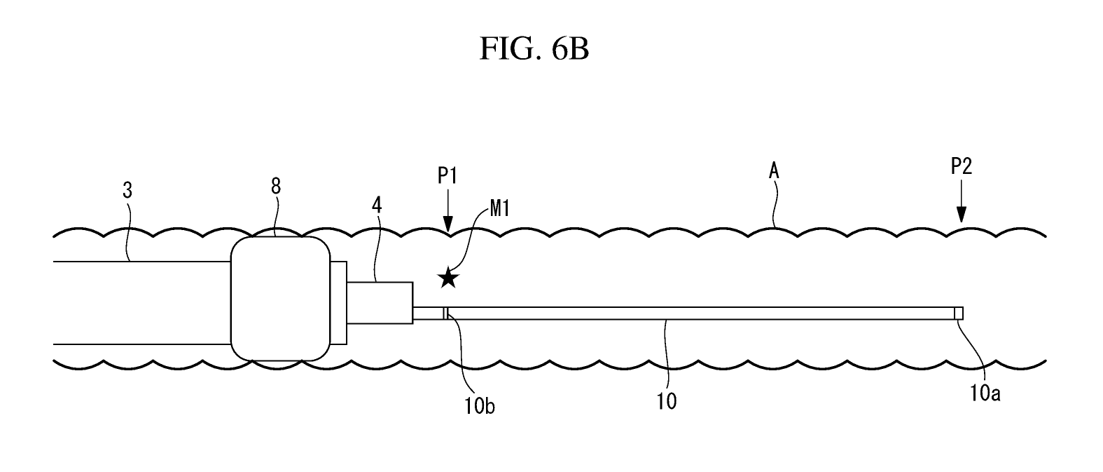

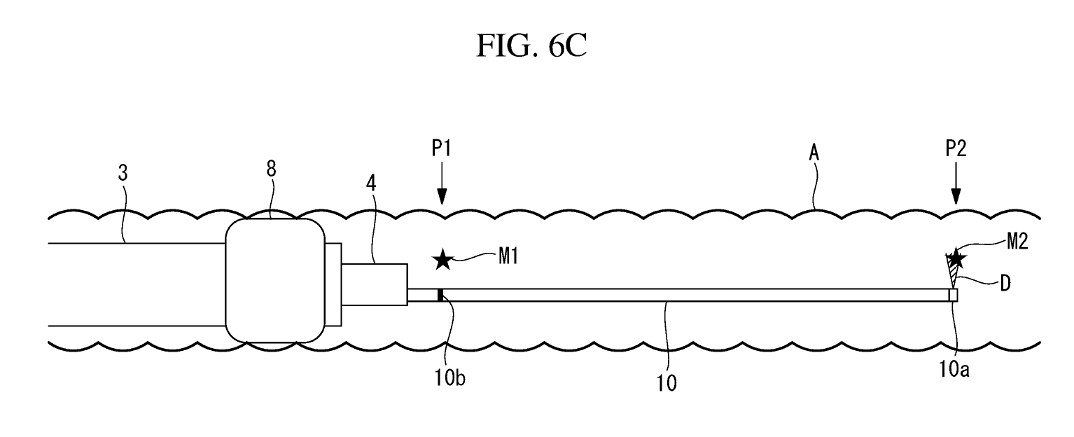

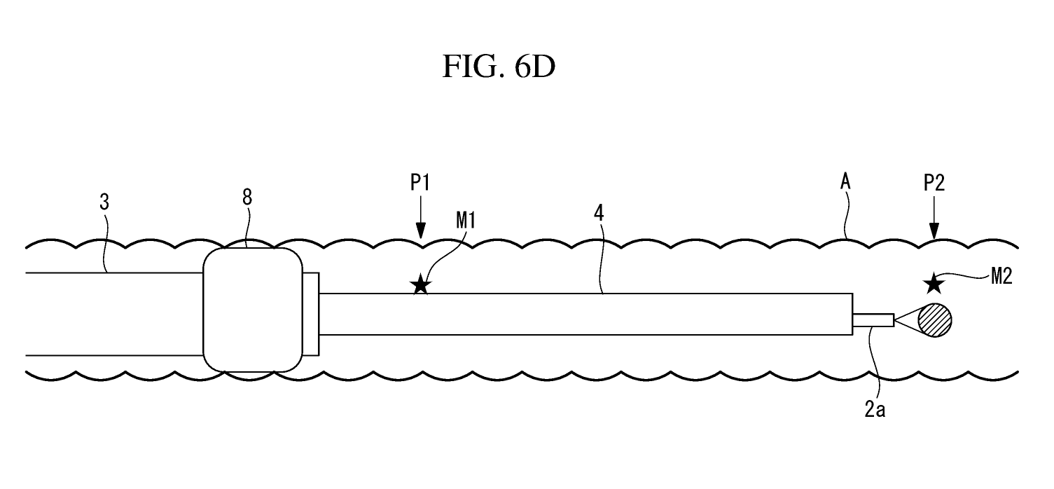

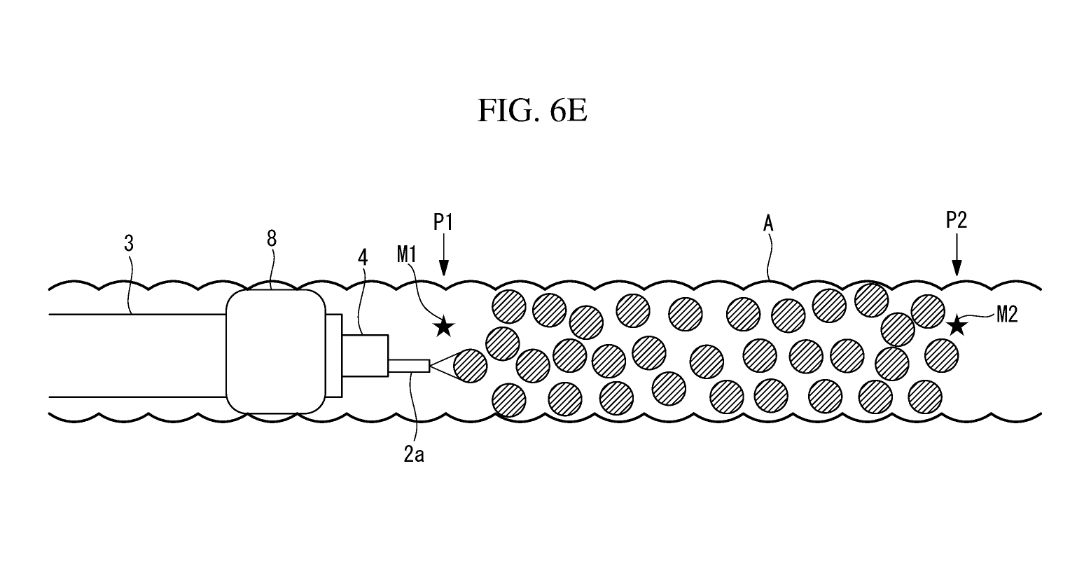

As shown in FIG. 6A, the marking apparatus 10 is inserted into the ileum A via the treatment tool channel 4a of the inserted portion 4. Next, while observing the end point P1 and the marking portion 10a, which is protruding from a distal-end opening of the treatment tool channel 4a in the endoscope image on the monitor 6, the dye D is sprayed from the marking portion 10a toward the ileal mucosa at the end point P1, thus forming a mark M1 with the dye D.

Next, in the start-point determining step S4, on the basis of the positioning mark 10b of the marking apparatus 10, the start point P2 is determined at a position that is separated from the end point P1 toward the jejunum by the length L. Specifically, as shown in FIG. 6B, while observing the mark M1 at the end point P1 by using the endoscope 1, the marking apparatus 10 is moved forward toward the jejunum, and the positioning mark 10b is positioned at the mark M1. By doing so, the marking portion 10a is positioned at the start point P2, which is separated from the end point P1 toward the jejunum by the length L.

Next, in the start-point marking step S5, as shown in FIG. 6C, the dye D is sprayed from the marking portion 10a toward the ileal mucosa at the start point P2, thus forming a mark M2 with the dye D.

As shown in FIG. 5B, at balloon 10c that can be inflated in radial directions may be provided at a distal end of the marking apparatus 10. The balloon 10c, in an inflated state, is fixed to the ileum A by being brought into contact with an inner wall of the ileum A. In the marking steps S3 and S5, by fixing the distal end of the marking apparatus 10 to the ileum A by means of the inflated balloon 10c, it is possible to more stably mark the end point P1 and the start point P2.

Next, in the ablation treatment step S6, ablation treatment is performed on the ileal mucosa by means of the ablation treatment tool 2.

Specifically, as shown in FIG. 6D, the ablation treatment tool 2 is inserted into the treatment tool channel 4a instead of the marking apparatus 10. Next, the inserted portion 4 is positioned at a position at which the mark M2 at the start point P2 is observed in the endoscope image, and the distal-end portion 2a of the ablation treatment tool 2 is made to protrude from the distal-end opening of the treatment tool channel 4a. Then, energy discharge from the distal-end portion 2a is started, and the ablation treatment is performed at the start point P2 of the ileal mucosa.

Subsequently, as shown in FIG. 6E, the ablation treatment by means of the distal-end portion 2a is repeated while changing the treatment positions of the distal-end portion 2a toward the ileocecal valve B by moving the ablation treatment tool 2 and the inserted portion 4 toward the base end. The ablation treatment is continued until reaching a position at which the mark M1 of the end point P1 is observed in the endoscope image. By doing so, the ablation treatment for the treatment area having the length L is completed.

Here, the relationship between suppression of the bile-acid absorption and metabolic disease will be described.

A part of the bile acid is newly synthesized from cholesterol in the liver, and the majority of the bile acid is recycled by enterohepatic circulation. Enterohepatic circulation refers to a cycle in which the bile acid secreted into the duodenum from the liver via the bile duct is returned to the liver via the jejunum, the ileum, and the portal-vein blood vessels. Bile acid has a known function of controlling glucose metabolism in various organs in the process of enterohepatic circulation. In other words, the amount and components of bile acid are closely involved in maintaining normal glucose metabolism. It is presumed that the amount and components of bile acid are effectively adjusted by adjusting the bile-acid absorption into blood vessels in the ileum.

The bile-acid absorption in the ileum is performed by the ASBT (Apical sodium-dependent bile acid transporter) present in the epithelium of the ileal mucosa. It is known that discharge of bile acid into the large intestine is promoted by partially removing the ileum, and that the degree of bile-acid discharge promotion changes in accordance with the excised length of the ileum. These phenomena occur due to a decrease in the bile-acid absorption efficiency caused by a loss of the bile-acid absorption region. In addition, administering a drug that inhibits bile-acid absorption (ASBT inhibitor or bile-acid sequestrant) to a diabetic patient has been reported to decrease the blood glucose level.

As a result of suppressing bile-acid absorption in the ileum by means of partial removal of the ileum or a drug, discharge of bile acid into the large intestine is promoted, and thus, the amount of bile acid to be recycled decreases. Bile acid newly synthesized in the liver compensates for the decrease in the amount of bile acid. The components of the bile acid recycled through the enterohepatic circulation and the components of the bile acid newly synthesized in the liver differ from each other. Therefore, when bile-acid absorption is suppressed, the compositions of the bile acid (the ratio of the recycled amount and newly synthesized amount of bile acid) change. It is presumed that improvement of diabetes due to the suppression of bile-acid absorption is achieved by adjusting the bile-acid compositions. This is consistent with the presumption that an improvement effect of the surgical bypass operation on obesity-complicated type-2 diabetes is achieved by adjusting the bile-acid pathway.

As with the inhibition of bile-acid absorption achieved by means of partial removal of the ileum and a drug, also by suppressing the ASBT function by means of ablation treatment of the ileal mucosa, the bile-acid compositions are adjusted by suppressing bile-acid absorption in the ileum A, and, by doing so, an improvement effect on diabetes is achieved. The degree of suppression of bile-acid absorption and the bile-acid compositions are controlled via the size of the treatment area in the ileum A.

With this embodiment, the length L corresponding to the desired size of the treatment area is set on the basis of at least one of the patient characteristics and pathophysiological conditions. Then, the end point P1 and start point P2 are determined at the two positions that are separated from each other by the length L, the end point P1 and the start point P2 are marked with the dye D, and the ablation treatment is performed on the ileal mucosa from the start point P2 to the end point P1. By doing so, it is possible to control the size of the treatment area in the ileal mucosa to an area of an appropriate size in accordance with the patient characteristics and pathophysiological conditions, and thus, there is an advantage in that it is possible to reliably achieve a desired therapeutic effect.

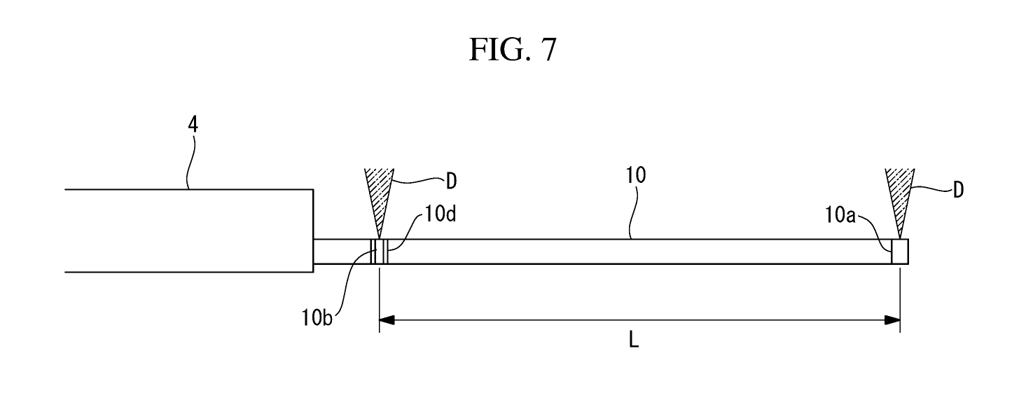

In this embodiment, although the marking apparatus 10 is moved after marking the end point P1 and the start point P2 is subsequently marked, alternatively, the end point P1 and the start point P2 may simultaneously be marked by using the marking apparatus 10 shown in FIG. 7.

The marking apparatus 10 in FIG. 7 includes another marking portion 10d provided at the position of the positioning mark 10b, and simultaneously sprays the dye D from the two marking portions 10a and 10d. In this case, the marking apparatus 10 is positioned at a position at which the positioning mark 10b is positioned at the end point P1, and, subsequently, the dye D is sprayed from the two marking portions 10a and 10d toward the end point P1 and the start point P2.

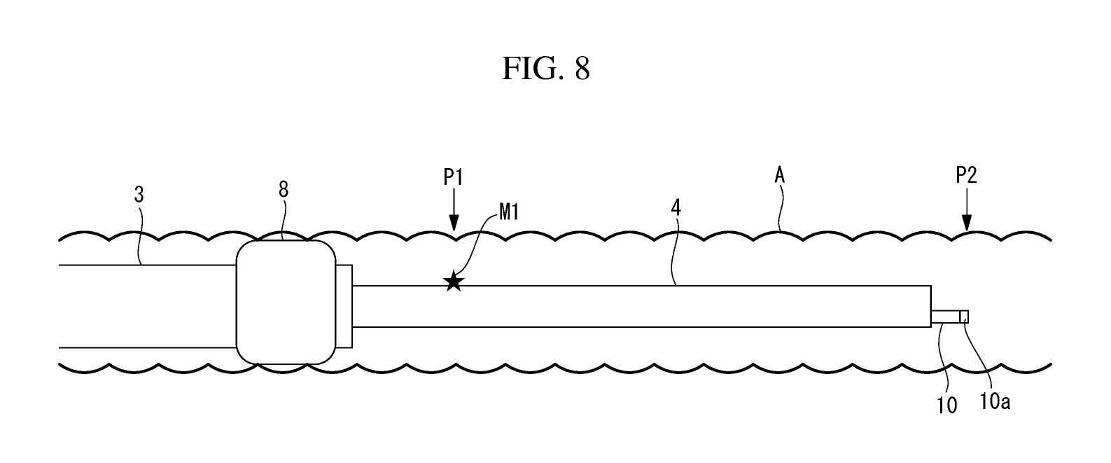

In this embodiment, although the start point P2 is determined by moving the marking apparatus 10 by an amount corresponding to the length L, alternatively, the start point P2 may be determined by moving the inserted portion 4 by an amount corresponding to the length L. Specifically, after marking the end point P1, as shown in FIG. 8, the inserted portion 4 and the marking apparatus 10 may integrally be moved forward toward the jejunum by an amount corresponding to the length L. By doing so, the marking portion 10a is positioned at the start point P2, which is separated from the end point P1 by the length L.

In this case, after determining the start point P2, the inserted portion 4 is positioned at a position at which the ileal mucosa at the start point P2 is observed in the endoscope image. Therefore, by exchanging the marking apparatus 10 in the treatment tool channel 4a with the ablation treatment tool 2 while maintaining the position of the inserted portion 4, it is possible to start ablation treatment from the start point P2 without having to mark the start point P2. The start-point marking step S5 may be omitted.

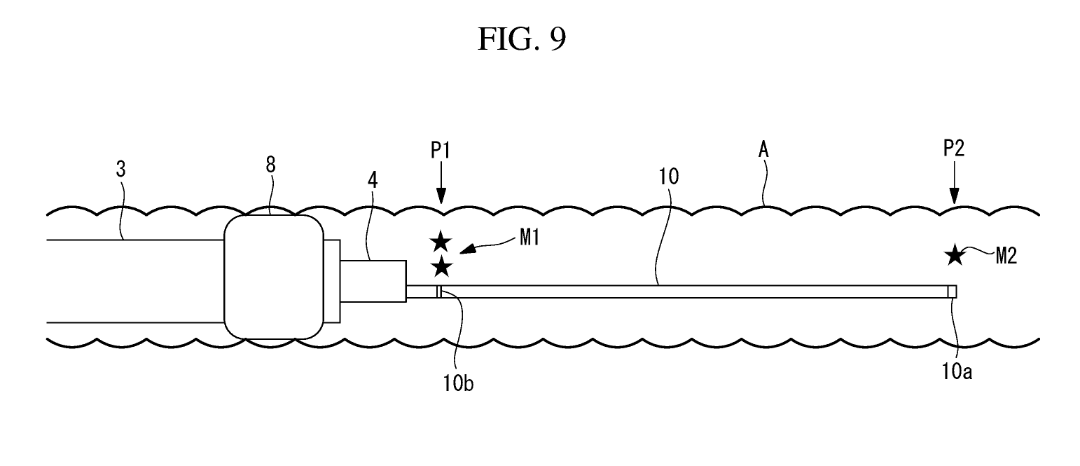

In this embodiment, the color of the mark M1 at the end point P1 and the color of the mark M2 at the start point P2 may be different from each other. Alternatively, as shown in FIG. 9, the pattern of the mark M1 and the pattern of the mark M2 may be different from each other. By making the appearances of the mark M1 and the mark M2 differ from each other in this way, it is possible to easily distinguish the end point P1 from the start point P2.

In this embodiment, although the end point P1 and the start point P2 are marked by using the dye D, alternatively, the end point P1 and the start point P2 may be marked by lightly treating the ileal mucosa with the ablation treatment tool 2.

In this case, because the work of exchanging the marking apparatus 10 in the treatment tool channel 4a with the ablation treatment tool 2 is omitted, it is possible to simplify the procedure.

In this embodiment, inserting the inserted portion 4 into the ileum A and confirming a treatable bile-acid absorption region on the basis of an endoscope image may be performed before the end-point determining step S2.

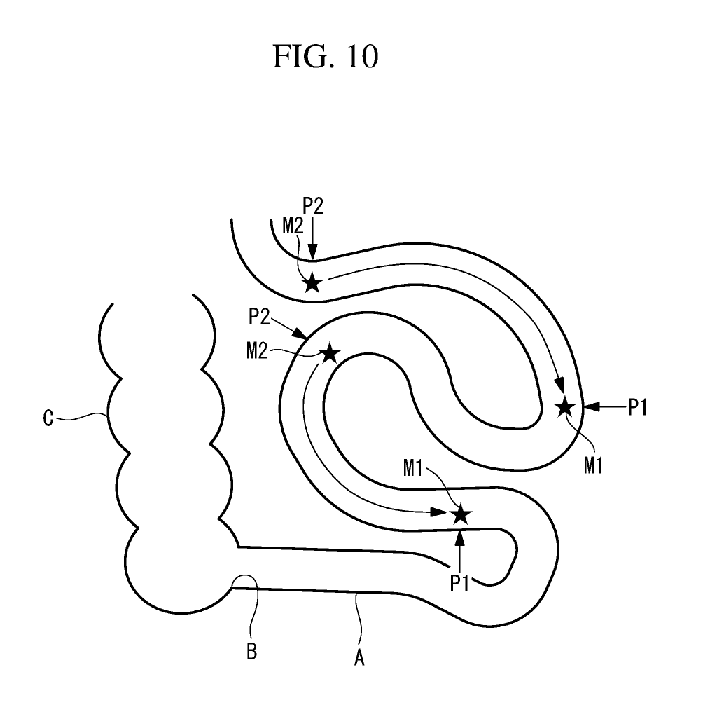

The small intestine includes locations at which it is difficult to perform treatment by means of the ablation treatment tool 2. For example, it is difficult to perform treatment at a portion that is bent at a large curvature. As shown in FIG. 10, the treatment area may be set so as to avoid locations at which it is difficult to perform the treatment. In other words, a treatment area may be divided into a plurality of areas, and the end point P1 and the start point P2 of each area may be marked.

The following aspects of the invention are derived from the above-described embodiment and modifications thereof.

An aspect of the present invention is a method for performing ablation treatment on an ileal mucosa that covers an inner surface of the ileum, the ileal-mucosa ablation treatment method comprising: setting a length of a treatment area in the ileal mucosa, wherein the length of the treatment area is set on the basis of at least one of patient characteristics and pathophysiological conditions; marking an end point of the treatment area in the ileal mucosa; and performing ablation treatment on the ileal mucosa, wherein, in the performing ablation treatment, ablation treatment is performed on the ileal mucosa from a start point to the end point while changing the treatment position toward the end point from the start point, and the start point is at a position that is separated from the end point in a longitudinal direction of the ileum by the set length.

With this aspect, ablation treatment is performed on the ileal mucosa from the start point to the end point of the treatment area after marking the end point of the treatment area. The length of the ileum from the start point to the end point is set on the basis of at least one of the patient characteristics and pathophysiological conditions. In addition, the operator can recognize the end point on the basis of the marking. Therefore, by performing ablation treatment on the treatment area from the start point to the end point, the treatment area in the ileal mucosa is appropriately controlled, and thus, it is possible to reliably achieve a desired therapeutic effect.

The above-described aspect may additionally include marking the start point in the ileal mucosa.

By employing this configuration, it is possible to recognize the start point.

In the above-described aspect, the marking the start point may be performed after the marking the end point, determining the start point may be additionally included between the marking the end point and the marking the start point, in the marking the end point, the end point may be marked by using a marking portion of an elongated marking apparatus inserted into the ileum, in the determining the start point, an positioning mark of the marking apparatus may be positioned at the marked end point by moving the marking apparatus in the longitudinal direction in the ileum, and the positioning mark may be provided at a position that is separated from the marking portion in the longitudinal direction of the marking apparatus by an amount corresponding to the length of the treatment area.

By moving the marking apparatus until the positioning mark is positioned at the marked end point after marking the end point by means of the marking portion, the marking portion is positioned at a position that is separated from the end point by the set length. Therefore, it is possible to determine the start point to be at the position of the moved marking portion.

In the above-described aspect, in the marking the end point and the marking the start point, the end point and the start point may be individually marked by two marking portions of the marking apparatus, and the two marking portions may be provided at positions that are separated from each other in the longitudinal direction of the marking apparatus by an amount corresponding to the length of the treatment area.

By employing this configuration, it is possible to easily mark the two positions that are separated from each other by the set length.

The above-described aspect may further comprise: determining the start point after the marking the end point, wherein, in the marking the end point, the end point may be marked by means of a marking portion of an elongated marking apparatus, the marking apparatus being inserted into the ileum via a treatment tool channel of an endoscope positioned in the ileum, and, in the determining the start point, the endoscope and the marking apparatus may be integrally moved, by an amount corresponding to the length of the treatment area, in the longitudinal direction in the ileum from the position at which the end point is marked.

By moving the endoscope and the marking apparatus by an amount corresponding to the length of the treatment area after marking the end point by means of the marking portion, the marking portion is positioned at a position that is separated from the end point by the length of the treatment area. Therefore, it is possible to determine the start point to be at the position of the moved marking portion. In addition, because the endoscope is positioned at the position at which it is possible to observe the start point, it is possible to start ablation treatment from the start point by using the ablation treatment tool inserted into the ileum via the treatment tool channel of the endoscope without having to mark the start point.

In the above-described aspect, in the marking the end point, the end point may be marked by means of a marking portion of a marking apparatus while fixing a distal-end portion of the marking apparatus to the ileal mucosa, and the distal-end portion of the marking apparatus may be fixed to the ileal mucosa by inflating a balloon provided at the distal-end portion of the marking apparatus.

The position of the marking portion is stabilized by fixing the distal-end portion of the marking apparatus to the ileal mucosa. Therefore, it is possible to more stably mark the end point.

### REFERENCE SIGNS LIST

100 ablation system
1 endoscope
2 ablation treatment tool
3 overtube
4 inserted portion
5 handle portion
6 monitor
7 controller
7a foot pedal
8 balloon
10 marking apparatus
10a, 10d marking portion
10b positioning mark
A ileum
B ileocecal valve
C large intestine
D dye
P1 end point
P2 start point

1. A method for performing ablation treatment on an ileal mucosa that covers an inner surface of the ileum, the ileal-mucosa ablation treatment method comprising:
setting a length of a treatment area in the ileal mucosa, wherein the length of the treatment area is set on the basis of at least one of patient characteristics and pathophysiological conditions;
marking an end point of the treatment area in the ileal mucosa; and
performing ablation treatment on the ileal mucosa,
wherein, in the performing ablation treatment, ablation treatment is performed on the ileal mucosa from a start point to the end point while changing the treatment position toward the end point from the start point, and the start point is at a position that is separated from the end point in a longitudinal direction of the ileum by the set length.

2. The ileal-mucosa ablation treatment method according to claim 1, further comprising:
marking the start point in the ileal mucosa.

3. The ileal-mucosa ablation treatment method according to claim 2,
wherein the marking the start point is performed after the marking the end point,
determining the start point is additionally included between the marking the end point and the marking the start point,
in the marking the end point, the end point is marked by using a marking portion of an elongated marking apparatus inserted into the ileum,
in the determining the start point, an positioning mark of the marking apparatus is positioned at the marked end point by moving the marking apparatus in the longitudinal direction in the ileum, and
the positioning mark is provided at a position that is separated from the marking portion in the longitudinal direction of the marking apparatus by an amount corresponding to the set length of the treatment area.

4. The ileal-mucosa ablation treatment method according to claim 2, wherein, in the marking the end point and the marking the start point, the end point and the start point are individually marked by two marking portions of the marking apparatus, and the two marking portions are provided at positions that are separated from each other in the longitudinal direction of the marking apparatus by an amount corresponding to the length of the treatment area.

5. The ileal-mucosa ablation treatment method according to claim 1, further comprising:
determining the start point after the marking the end point,
wherein, in the marking the end point, the end point is marked by means of a marking portion of an elongated marking apparatus, the marking apparatus being inserted into the ileum via a treatment tool channel of an endoscope disposed in the ileum, and,
in the determining the start point, the endoscope and the marking apparatus are integrally moved, by an amount corresponding to the length of the treatment area, in the longitudinal direction in the ileum from the position at which the end point is marked.

6. The ileal-mucosa ablation treatment method according to claim 1,
wherein, in the marking the end point, the end point is marked by means of a marking portion of a marking apparatus while fixing a distal-end portion of the marking apparatus to the ileal mucosa, and
the distal-end portion of the marking apparatus is fixed to the ileal mucosa by inflating a balloon provided at the distal-end portion of the marking apparatus.
